(19) United States
(12) Patent Application Publication
(10) Pub. No.: US 2022/0000869 A1
(43) Pub. Date: Jan. 6, 2022

(54) USE OF SHP2 INHIBITORS FOR THE TREATMENT OF INSULIN RESISTANCE

(71) Applicants: INSERM (INSTITUT NATIONAL DE LA SANTÉ ET DE LA RECHERCHE MÉDICALE), Paris (FR); UNIVERSITÉ PAUL SABATIER TOULOUSE III, Toulouse (FR)

(72) Inventors: Armelle YART, Toulouse Cedex 4 (FR); Romain PACCOUD, Boston, MA (US); Mylène TAJAN, London (GB); Jean-Philippe PRADERE, Toulouse Cedex 4 (FR); Philippe VALET, Toulouse Cedex 4 (FR); Cédric DRAY, Toulouse Cedex 4 (FR)

(21) Appl. No.: 17/295,600

(22) PCT Filed: Nov. 22, 2019
(86) PCT No.: PCT/EP2019/082180

§ 371 Date: May 20, 2021

(30) Foreign Application Priority Data
Nov. 23, 2018 (EP) 18306558.0

(51) Int. Cl. A61K 31/506; A61P 3/10

(52) U.S. Cl. CPC A61K 31/506; A61P 3/10

(57) ABSTRACT

Despite reaching an epidemic status worldwide, metabolic disorders, notably diabetes, still miss efficient and specific therapeutic strategies because of their multifactorial origin. SHP2 is a ubiquitous tyrosine phosphatase that regulates major signalling pathways (e.g. MAPK, PI3K) in response to many growth factors. The inventors evaluate whether chronic inhibition of SHP2 could improve insulin sensitivity in animal models. Obese diabetic mice were thus treated by gavage (50 mg/kg/day). And the inventors note a significant improvement in the glucose tolerance of the treated animals compared to their control, with a decreased fasting blood glucose, without any change in weight or body composition. Accordingly, the present invention relates to use of SHP2 inhibitors for the treatment of insulin resistance.

### FIELD OF THE INVENTION

The present invention relates to use of SHP2 inhibitors for the treatment of insulin resistance.

### BACKGROUND OF THE INVENTION

Despite reaching an epidemic status worldwide, metabolic disorders, notably diabetes, still miss efficient and specific therapeutic strategies because of their multifactorial origin. Resistance of peripheral tissues (liver, muscle, adipose tissue) to insulin action is a key event in diabetes onset, so that therapeutic strategies aiming at restoring insulin sensitivity are highly relevant. However, they essentially remain unsatisfactory. For instance, thiazolidinediones, by binding peroxisome proliferator activated receptor gamma, increase insulin sensitivity, but such treatments are associated to important side effects. There is therefore still an important need to understand insulin resistance-promoting mechanisms to identify therapeutic targets.

SHP2 is a ubiquitous tyrosine phosphatase that regulates major signalling pathways (e.g. MAPK, PI3K) in response to many growth factors, thereby having key functions during development, so that its dysregulation has been linked to developmental disorders as well as cancers (Tajan, M., de Rocca Serra, A., Valet, P., Edouard, T., and Yart, A. (2015). SHP2 sails from physiology to pathology. European journal of medical genetics 58, 509-525.). SHP2 also plays major roles in metabolism regulation, as evidenced by the severe phenotype of its targeted invalidation in various tissues and organs, however the global metabolic role of SHP2, and the contribution of its deregulation to metabolic diseases, have never been ascertained (Tajan, M., de Rocca Serra, A., Valet, P., Edouard, T., and Yart, A. (2015). SHP2 sails from physiology to pathology. European journal of medical genetics 58, 509-525.). Several SHP2 inhibitors have been described in the prior art (e.g. WO2010121212 and WO2015003094) but their use in the treatment of insulin resistance has never been described. In contrast, the concept of using strategies aiming at activating SHP2 to alleviate obesity and or diabetes has been proposed, based on the identification of a primary function of SHP2 in postmitotic forebrain neurons is the control of energy balance and metabolism (Zhang E E, Chapeau E, Hagihara K, Feng G S. Neuronal Shp2 tyrosine phosphatase controls energy balance and metabolism. Proc Natl Acad Sci USA. 2004 Nov. 9; 101(45):16064-9). Moreover, mice with a neuron-specific, conditional Shp2 deletion, developed obesity and diabetes and the associated pathophysiological complications that resemble those encountered in humans, including hyperglycemia, hyperinsulinemia, hyperleptinemia, insulin and leptin resistance, vasculitis, diabetic nephropathy, urinary bladder infections, prostatitis, gastric paresis, and impaired spermatogenesis (Krajewska M 1, Banares S, Zhang E E, Huang X, Scadeng M, Jhala U S, Feng G S, Krajewski S Development of diabesity in mice with neuronal deletion of Shp2 tyrosine phosphatase. Am J Pathol. 2008 May; 172(5):1312-24.).

### SUMMARY OF THE INVENTION

The present invention relates to use of SHP2 inhibitors for the treatment of insulin resistance. In particular, the present invention is defined by the claims.

### DETAILED DESCRIPTION OF THE INVENTION

The first object of the present invention relates to a method of treating insulin resistance in a subject in need thereof comprising administering to the subject a therapeutically effective amount of a SHP2 inhibitor.

As used herein, the term “subject” refers to a human or another mammal (e.g., primate, dog, cat, goat, horse, pig, mouse, rat, rabbit, and the like), that can be afflicted with lupus. In a particular embodiment of the present invention, the subject is a human being. In such embodiments, the subject is often referred to as an “individual”. The term “individual” does not denote a particular age, and thus encompasses children, teenagers, and adults.

As used herein, the term “insulin resistance” has its common meaning in the art. Insulin resistance is a physiological condition where the natural hormone insulin becomes less effective at lowering blood sugars. The resulting increase in blood glucose may raise levels outside the normal range and cause adverse health effects such as metabolic syndrome, dyslipidemia and subsequently type 2 diabetes mellitus. The method of the present invention is thus particularly suitable for the treatment of type 2 diabetes. As used herein, the term “type 2 diabetes” or “non-insulin dependent diabetes mellitus (NIDDM)” has its general meaning in the art. Type 2 diabetes often occurs when levels of insulin are normal or even elevated and appears to result from the inability of tissues to respond appropriately to insulin. Most of the type 2 diabetics are obese.

In some embodiments, the subject suffers from obesity. As used herein the term “obesity” refers to a condition characterized by an excess of body fat. The operational definition of obesity is based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject with at least one obesity-induced or obesity-related co-morbidity that requires weight reduction or that would be improved by weight reduction, has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m² to less than 25 kg/m².

As used herein, the term “treatment” or “treat” refer to both prophylactic or preventive treatment as well as curative or disease modifying treatment, including treatment of subject at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]). In particular, the method of the present invention is particularly suitable for improving blood glucose control, enhancing insulin signalling in skeletal muscle and adipose tissue, reducing lipotoxicity in skeletal muscle and adipose tissue, increasing lipid oxidative capacity in skeletal muscle and adipose tissue, or maintaining long-term insulin sensitivity in the subject.

As used herein, the term “SHP2” has its general meaning in the art and refers to the protein encoded by the PTPN11 gene. SHP2 is a non-receptor protein tyrosine phosphatase (PTP) with two Src homology-2 (SH2) domains (N-SH2, C-SH2) (Alonso et. al., 2004; Neel et al., 2003). SHP2 is also known as Homo sapiens protein tyrosine phosphatase, non-receptor type 11 (PTPN11).

As used herein, a “SHP2 inhibitor” refers to any compound natural or not which is capable of inhibiting the activity of SHP2, in particular SHP2 phosphatase activity. SHP2 inhibitors are well known in the art. The term encompasses any SHP2 inhibitor that is currently known in the art or that will be identified in the future. The term also encompasses inhibitor of expression. In some embodiments, the SHP2 inhibitor is selective over the other phosphatases including SHP1. By “selective” it is meant that the inhibition of the selected compound is at least 10-fold, preferably 25-fold, more preferably 100-fold, and still preferably 300-fold higher than the inhibition of the other phosphatases. Typical assays are also described in WO2010121212 and WO2015003094. Typically, the SHP2 inhibitor is a small organic molecule.

Non-limiting examples of SHP2 inhibitors include NSC-87877 (also known as 8-Hydroxy-7-[(6-sulfo-2-naphthyl)azo]-5-quinolinesulfonic acid), estradiol phosphate, estramustine phosphate, PHPS1, NSC-117199, SP1-112, SP1-112Me (and see Chen, L. et al., 2006 and Chen, L. et al., 2010), tautomycetin analogs (e.g., see Liu, S. et al., 2011), phenylhydrazonopyrazolone sulfate and compounds described in Hellmuth, K. et al., 2008, compounds described in United States Patent Application Publication No. 20120034186 (U.S. Ser. No. 13/274,699) and compounds described in Yu, Z. H. et al. 2011.

In some embodiments, the SHP2 inhibitor for use according to the present invention is selected from compounds described in WO2010121212, WO2015003094, WO2017100279, and WO2007117699.

In some embodiments, the SHP2 inhibitor for use according to the present invention is 4,4′-(4′-Carboxy)-4-nonyloxy-[1,1′-biphenyl]-3,5-diyl)dibutanoic acid.

In some embodiments, the SHP2 inhibitor for use according to the present invention is SHP099: 6-(4-amino-4-methylpiperidin-1-yl)-3-(2,3-dichlorophenyl)pyrazin-2-amine (Garcia Fortanet J, Chen C H, Chen Y N, Chen Z, Deng Z, Firestone B, Fekkes P, Fodor M, Fortin P D, Fridrich C, Grunenfelder D, Ho S, Kang Z B, Karki R, Kato M, Keen N, LaBonte L R, Larrow J, Lenoir F, Liu G, Liu S, Lombardo F, Majumdar D, Meyer M J, Palermo M, Perez L, Pu M, Ramsey T, Sellers W R, Shultz M D, Stams T, Towler C, Wang P, Williams S L, Zhang J H, LaMarche M J. Allosteric Inhibition of SHP2: Identification of a Potent, Selective, and Orally Efficacious Phosphatase Inhibitor. J Med Chem. 2016 Sep 8; 59(17):7773-82).

In some embodiments, the SHP2 inhibitor for use according to the present invention is a compound having a formula (I);

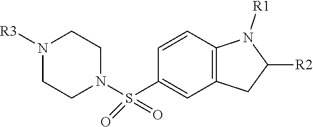

wherein R₁ is selected from the group consisting of carboalkoxy, benzylcarboxamide, straight chained, branched or cyclic alkyl, CO(CH₂)₂CO₂H, COCH₂CH(OH)CO₂H, CO(CH)₂CO₂H, CONH(C₆H₄)CI, CONH(C₆H₄)CH(CH₃)₂, CONH(C₆H₄)(CI)₂, CO(CH₂)₃CO₂H, CO(C₆H₄)OCH₃, CO(C₅H₈), CO(C₆H₄)CI, CONHCH₂CO₂H, COCH₂CH(CH₃)₂, CONHCH(CHCH₃CH₂CH₃)(CO₂H), CONH(C₆H₄)OCH₃, CO(C₆H₃)(CI)₂, COCH₂(C₆H₄)CI, CO(CH₂)₂CO₂H, CONH(C₆H₄)F, CONHCH(CH₃)CO₂H, CO(C₃H₅), CON HCH(CH₂CHCH₃CH₃)(CO₂CH₂CH₃), CO(C₆H₅), CONHCH(CHCH₃CH₃)(CO₂H), COCH₂CH(OH)(CO₂″)—Na⁺, CONHCH(CH₃)(CO₂CH₂CH₃), CONH(C₆H₄)CF₃, COCO₂H, CO(CH₂)₂CH₃, CONH(CH₂)₂CO₂H, CONHCH(CHCH₃CH₃)(CO₂CH₂CH₃), CONHCH(CH₂CHCH₃CH₃)(CO₂H), CONH(C₆H₅), CONH(CH₂)₂CO₂CH₂CH₃, CONHCH(CH₂C₆H₅)(CO₂CH₂CH₃), CONHCH(CHCH₃CH₂CH₃)(CO₂CH₃), CONH(C₆H₄)CO₂H, CO(C₆H₈)CO₂H, CONHCH(CH₂C₆H₅)(CO₂H), CONH(C₆H₄)CO₂CH₃, COCH₂OCH₃, COCH₂(C₆H₅), COCF₃, CONHCH(CH₂CH₂CO₂H)(CO₂H), and CON HCH₂CO₂CH₂CH₃;
wherein R₂ is selected from the group consisting of H, and CH₃; and wherein R₃ is selected from the group consisting of (C₆H₄)CI, (C₆H₄)CO₂H, (C₆H₄)CH₃, (C₆H₄)(CI)₂, (C₆H₄)(CF₃)₂,
(C₆H₄)OCH₃, (C₆H₄)F, (C₆H₃)(CI)(CH₃), and C₆H₅.

In some embodiments, R1 is selected from the group consisting of COCH2CH2COOH and COCH2CH(OH)CO2H.

In some embodiments R1 is selected from the group consisting of CO(CH)2CO2H, CONH(C6H4)CH(CH3)2, CO(CH2)3CO2H, CONHCH2CO2H, CO(CH2)2CO2H, CONHCH(CHCH3CH3)(CO2H), COCO2H, CONH(CH2)2CO2H, and CONHCH(CH2CHCH3CH3)(CO2H).

In some embodiments, R1 is CO(CH2)2CO2H, R2 is H, and R3 is (C6H4)CI.

In some embodiments, the SHP2 inhibitor for use according to the present invention is a compound having a formula (IV):

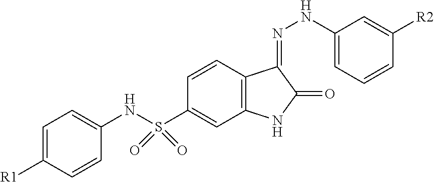

wherein R₁ is selected from the group consisting of F; and wherein R₂ is selected from the group consisting of COOCH₃, and CO₂″ ▪N⁺H₂(CH₃)(CH₂CHOH)₄CH₂OH).

In some embodiments, the SHP2 inhibitor for use according to the present invention is selected from the group consisting of:

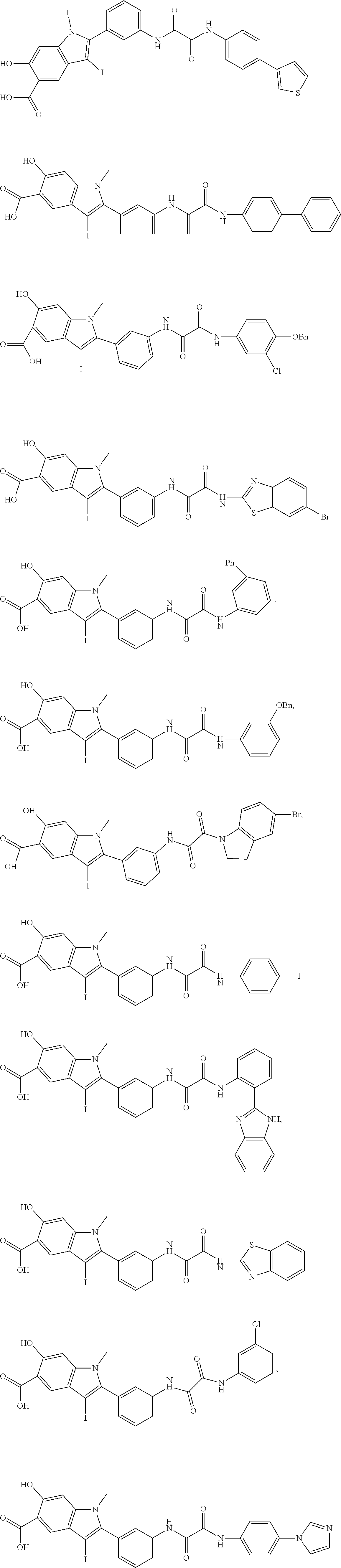

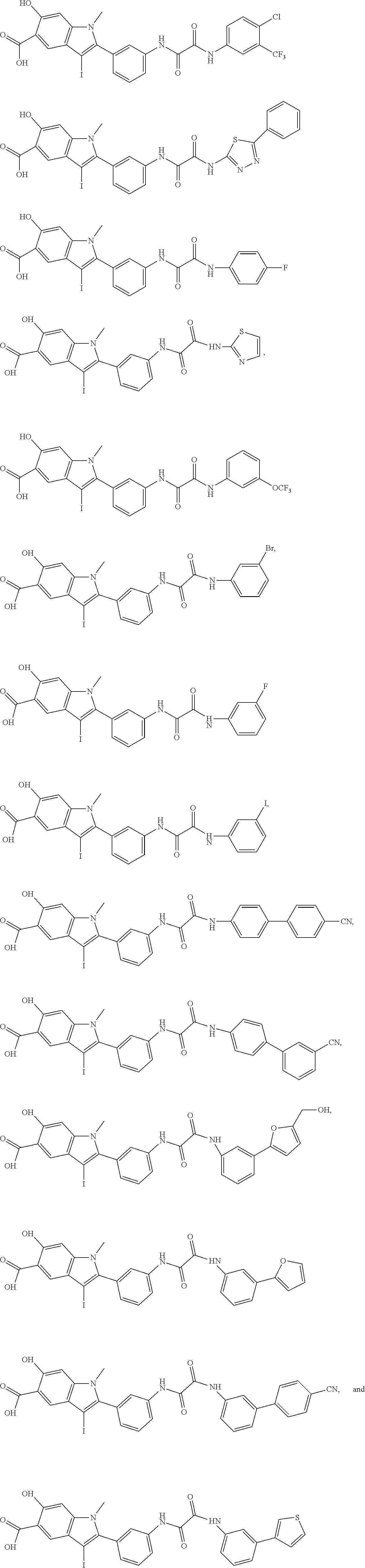

In some embodiments, the SHP2 inhibitor is an inhibitor of SHP2 expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of SHP2 mRNA by binding thereto and thus preventing protein translation or increasing mRNA degradation, thus decreasing the level of SHP2, and thus activity, in a cell. For example, antisense oligonucleotides of at least about 15 bases and complementary to unique regions of the mRNA transcript sequence encoding SHP2 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. SHP2 gene expression can be reduced by contacting a subject or cell with a small double stranded RNA (dsRNA), or a vector or construct causing the production of a small double stranded RNA, such that SHP2 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing SHP2. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Ban viruses; papilloma viruses; herpes virus; vaccinia virus; polio virus; and RNA virus such as a retrovirus/lentivirus. One can readily employ other vectors not named but known to the art.

In some embodiments, the SHP2 inhibitor is embedded or conjugated to a nanoparticule, so that the SHP2 inhibitor would be preferentially deliver in myeloid cells (e.g. macrophages) that are able to phagocyte said nanoparticle. As used herein, the term “nanoparticle” encompasses liposomes, polymer micelles, polymer-DNA complexes (polycomplexes), nanospheres, nanofibres, nanotubes, and nanocapsules. All these nanoparticles are known in the art. The surface of such nanoparticles is often modified by PEG brush (PEGylation, i.e. polyethylene glycol (PEG) is attached to the surface of the nanoparticles). In some embodiments, the nanoparticle is a nanocapsule. As used herein, the term “nanocapsules” means vesicular systems in which the drug is confined to a cavity surrounded by a uniquer polymer membrane. In some embodiments, the nanoparticle is a nanosphere. As used herein, the term “nanosphere” means a matrix system in which the drug is physically and uniformly dispersed. In some embodiments, the nanoparticle is a liposome. As used herein, the term “liposome” includes any structure composed of a lipid bilayer that enclose one or more volumes, wherein the volume can be an aqueous compartment. Liposome consist of one, two, three, four, five, six, seven, eight, nine, ten or more lipid bilayers. The term “lipid bilayer” includes, but is not limited to: phospholipid bilayer, bilayer consisting of nonionic surfactants. Liposomes consisting of a phospholipid bilayer can be composed of naturally-derived phospholipids with mixed lipid chains (like e.g. phosphatidylethanolamine), or of pure components like DOPE (dioleolylphosphatidyl-ethanolamine) but are not limited to these components. Liposomes include—but are not limited to—emulsions, foams, micelles, exosomes, vesicles, insoluble monolayers, liquid crystals, phospholipid dispersions, lamellar layers and the like. The term “liposome” also includes so called “stealth liposomes” which consist of water-soluble polymers (e.g. polyethyleneglycol, PEG) attached to the surface of conventional liposomes composed of a lipid mono- or bilayer that enclose a volume (e.g. so called PEGylated liposomes). Following liposome preparation, the liposomes may be sized to achieve a desired size range and relatively narrow distribution of liposome sizes. Methods of coupling inhibitors according to the present invention to liposomes generally involve either covalent cross linking between a liposomal lipid and an inhibitor. In another approach, an inhibitor according to the present invention has been covalently derivatized with a hydrophobic anchor, such as fatty acids, is incorporated into a preformed lipid.

According to the invention, the SHP2 inhibitor is administered to the subject in a therapeutically effective amount. By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the active ingredient of the present invention (e.g. SHP2 inhibitor) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1 shows that obese diabetic mice treated by gavage with a SHP2 inhibitor (50 mg/kg/day) have significant improvement in the glucose tolerance.

### EXAMPLE

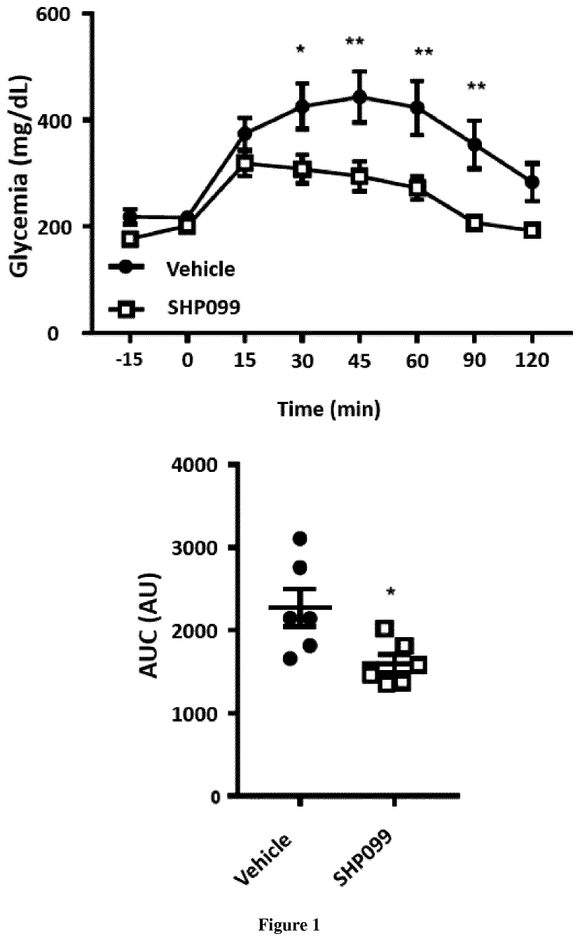

Several specific inhibitors of SHP2 have been developed, particularly in the field of anti-cancer therapies. One of the compound is SHP099 (6-(4-amino-4-methylpiperidin-1-yl)-3-(2,3-dichlorophenyl)pyrazin-2-amine) that has recently been shown to be being highly specific, with good tolerance and oral bioavailability (Garcia Fortanet J, Chen C H, Chen Y N, Chen Z, Deng Z, Firestone B, Fekkes P, Fodor M, Fortin P D, Fridrich C, Grunenfelder D, Ho S, Kang Z B, Karki R, Kato M, Keen N, LaBonte L R, Larrow J, Lenoir F, Liu G, Liu S, Lombardo F, Majumdar D, Meyer M J, Palermo M, Perez L, Pu M, Ramsey T, Sellers W R, Shultz M D, Stams T, Towler C, Wang P, Williams S L, Zhang J H, LaMarche M J. Allosteric Inhibition of SHP2: Identification of a Potent, Selective, and Orally Efficacious Phosphatase Inhibitor. J Med Chem. 2016 Sep. 8; 59(17):7773-82). A very recent study has also demonstrated its effectiveness in treatment of renal fibrosis induced by carbon tetrachloride (Kostallari et al., 2018). We therefore, evaluate whether chronic inhibition of SHP2 improves insulin sensitivity in animal models. Obese diabetic mice were treated by gavage (50mg/kg/day). After 15 days of treatment an OGTT was performed (FIG. 1). We note a significant improvement in the glucose tolerance of the treated animals compared to their control, with a decreased fasting blood glucose, without any change in weight or body composition.

### REFERENCES

Throughout this application, various references describe the state of the art to which this invention pertains. The disclosures of these references are hereby incorporated by reference into the present disclosure.

1. A method of treating insulin resistance in a subject in need thereof comprising administering to the subject a therapeutically effective amount of a SHP2 inhibitor.

2. The method of claim 1 wherein the subject suffers from type 2 diabetes.

3. The method of claim 1 wherein the subject suffers from obesity.

4. The method of claim 1 wherein the SHP2 inhibitor is a small organic molecule.

5. The method of claim 1 wherein the SHP2 inhibitor is SHP099 (6-(4-amino-4-methylpiperidin-1-yl)-3-(2,3-dichlorophenyl)pyrazin-2-amine).

6. The method of claim 1 wherein the SHP2 inhibitor is an inhibitor of SHP2 expression.

7. The method of claim 6 wherein the inhibitor of gene expression is a siRNA, an antisense oligonucleotide or a ribozyme.
